(19) United States
(12) Patent Application Publication
(10) Pub. No.: US 2025/0027118 A1
(43) Pub. Date: Jan. 23, 2025

(54) PHENYL ALCOHOLS FROM GAS FERMENTATION

(71) Applicant: LanzaTech, Inc., Skokie, IL (US)

(72) Inventors: Sean Alex Rollag, Chicago, IL (US); Brian Christopher Vicente, Chicago, IL (US); Taylor Craig Schulz, Chicago, IL (US)

(21) Appl. No.: 18/775,468

(22) Filed: Jul. 17, 2024

Related U.S. Application Data
(60) Provisional application No. 63/527,381, filed on Jul. 18, 2023.

(51) Int. Cl. C12P 7/22; B01D 3/10

(52) U.S. Cl. CPC C12P 7/22; B01D 3/10

(57) ABSTRACT

Process for separation of phenyl alcohol fermentation product from fermentation broth by employing both distillation and phase separation where the phenyl alcohol is removed from a distillation column as a side draw. Process provides a less complex and less costly alternative to conventional separation techniques used for high boiling point materials from fermentation broth.

### CROSS REFERENCE TO RELATED APPLICATIONS

This application claims the benefit of U.S. Provisional Patent Application No. 63/527,381 filed on Jul. 18, 2023, the entirety of which is incorporated herein by reference.

### FIELD

This disclosure relates to a process separating and purifying phenyl alcohols produced by fermentation. In particular, this disclosure relates to the use of a specific configuration of distillation to separate phenyl alcohols from other fermentation products and fermentation media.

### BACKGROUND

The following discussion is provided to aid the reader in understanding the disclosure and is not admitted to describe or constitute prior art thereto.

Fermentation in general, and gas fermentation processes in particular can be used to generate target materials from gas substrates or other input materials, particularly carbon-based materials. For example, particular biological systems can be used to perform gas fermentation.

Mitigation of impending climate change requires drastic changes in manufacturing and greater reliance on biotechnology. Sustainable sources of fuels and chemicals are currently insufficient to significantly displace dependence on fossil carbon. Biotechnology harnesses the power of biology to create new products in a way that improves the quality of life and the environment, Gas fermentation is emerging as a powerful biotechnological advancement as an alternative platform for the biological fixation of such gases such as CH₄, CO, CO₂, and/or H₂ into sustainable fuels and chemicals. In particular, gas fermentation technology can utilize a wide range of feedstocks including gasified carbon-containing matter such as municipal solid waste or agricultural waste, or industrial waste gases such as off-gases from steel manufacturing, petroleum refineries, and petrochemical processes to produce ethanol, aviation fuel, chemicals, and a variety of other products. Gas fermentation alone could displace 30% of crude oil use and reduce global CO₂ emissions by 10%. As with any disruptive technology, many technical challenges must be overcome before this potential is fully achieved. The science of scale-up production and the reduction of obstacles for continued commercialization of gas fermentation are advanced by this disclosure.

Gas fermentation involves a gas stream of carbon monoxide, carbon dioxide, and or hydrogen provided to a bioreactor housing a microorganism biocatalyst. Although industrial processes can output gases that have significant amounts of carbon-based materials such as carbon dioxide, many other sources of carbon are available. Solid and liquid sources of carbon may be processed in a feedstock preparation process to provide synthesis gas, commonly called syngas which may in turn become feedstock to the gas fermentation process. The feedstock preparation process, which is processing to provide syngas, involves partial oxidation, torrefaction, pyrolysis, or gasification.

Desired fermentation products need to be separated from the fermentation broth and recovered. Often this separation is a multi-step process where product(s) are separated, and biocatalyst and media are recycled to the bioreactor.

There exists a need to efficiently separate phenyl alcohols, for example, 2-phenylethanol (2-PE), from effluent of a fermentation operation. Efficient separation increases overall value of the process. Separation of phenyl alcohols is challenging due to the high boiling point of phenyl alcohols. Current fermentation operations produce low concentrations of phenyl alcohol in the fermentation effluent, which is difficult to separate from the rest of the fermentation effluent, with or without biocatalyst, as large amounts of water must be lifted overhead in distillation to remove a distillation bottoms stream comprising phenyl alcohol. Traditionally, after distillation, the distillation bottoms stream also needs to be purified by another technique such as simulated moving bed separation (SMB) to remove byproducts and other fermentation components. However, SMB operations are complex and costly and is complicated by the presence of biocatalyst and other fermentation products or components found in the fermentation effluent.

### SUMMARY

In a first aspect, the present disclosure provides a process comprising: (a) generating a fermentation broth comprising microbial biomass, at least one phenyl alcohol fermentation product, at least one byproduct, and an aqueous liquid nutrient medium; (b) removing the microbial biomass from the fermentation broth to generate a microbial biomass-depleted stream; (c) distilling, in a distillation column, the microbial biomass-depleted stream and removing water in a distillation bottoms stream, the at least one byproduct in a distillation overhead stream, and the at least one phenyl alcohol in a distillation side draw stream; (d) settling the side draw stream and forming at least two phases wherein a first phase comprises aqueous liquid nutrient medium and a second phase comprising phenyl alcohol; and (e) recovering the second phase comprising phenyl alcohol.

In a second aspect, the present disclosure provides a process comprising: (a) generating a fermentation broth comprising microbial biomass, at least one phenyl alcohol fermentation product, at least one byproduct, and an aqueous liquid nutrient medium; (b) removing the microbial biomass from the fermentation broth by vacuum distillation to generate a microbial biomass-depleted distillate; (c) settling the distillate and forming at least two phases wherein a first phase comprises aqueous liquid nutrient medium and a second phase comprising phenyl alcohol (c) distilling, in a distillation column, the second phase and removing water in a distillation bottoms stream, the at least one byproduct in a distillation overhead stream, and the at least one phenyl alcohol in a distillation side draw stream; and (e) recovering the second phase comprising phenyl alcohol.

It is an object of the present disclosure to provide one or more process which goes at least some way towards overcoming or ameliorating at least one of the disadvantages of the prior art. This object, and any other objectives or advantages or the like referred to herein or taken from this description, are to be read disjunctively and with the alternative object of to at least provide the public with a useful choice.

### BRIEF DESCRIPTION OF THE DRAWINGS

The figures has been simplified by the deletion of a large number of apparatuses customarily employed in a process/system of this nature, such as vessel internals, temperature and pressure control systems, flow control valves, recycle pumps and the like, which are not specifically required to illustrate the performance of the disclosure. Furthermore, the illustration of the process of this disclosure in the embodiment of a specific drawing is not intended to limit the disclosure to specific embodiments. Some embodiments may be described by reference to the process configuration shown in the Figure, which relates to both apparatus and processes to carry out the disclosure. Any reference to a process step includes reference to an apparatus unit or equipment that is suitable to carry out the step, and vice-versa.

FIG. 1 is a simplified overview if the piping and associated components of an embodiment of the process, particularly when the fermentation is a gas fermentation operation, in accordance with one embodiment of the disclosure.

FIG. 2 is a simplified schematic process flow diagram showing one embodiment of the disclosure, and focusing on a product recovery zone.

FIG. 3 is a simplified schematic process flow diagram showing one embodiment of the disclosure, and focusing on a distillation column in a product recovery zone.

FIG. 4 is a simplified schematic process flow diagram showing one embodiment of the disclosure, showing a particular order of units in a product recovery zone.

FIG. 5 is a simplified schematic process flow diagram showing one embodiment of the disclosure, showing a particular order of units in a product recovery zone.

FIG. 6 is a simplified schematic process flow diagram showing one embodiment of the disclosure, showing a particular order of units in a product recovery zone.

FIG. 7 is a plot of simulated modeling data in accordance with Example 2.

FIG. 8 is a plot of simulated modeling data in accordance with Example 2.

FIG. 9 is a plot of simulated modeling data in accordance with Example 3.

FIG. 10 is a plot of simulated modeling data in accordance with Example 3.

FIG. 11 is a plot of simulated modeling data in accordance with Example 7.

FIG. 12 is a plot of simulated modeling data in accordance with Example 7.

FIG. 13 is a plot of simulated modeling data in accordance with Example 7.

FIG. 14 is a plot of simulated modeling data in accordance with Example 7.

### DETAILED DESCRIPTION

The present disclosure provides processes and systems for improving value of fermentation processes by providing s technique of separation of fermentation product phenyl alcohols. Such separated and potentially purified phenyl alcohols may be an intermediate in other chemical reactions, used in manufacturing operations, or may be used as an ingredient in an article of manufacture. For example, 2-PE is a compound that is traditionally used as a fragrance. It can be converted to molecules like styrene, phenylacetaldehyde, phenylacetic acid, and benzyl cyanide. These can then be used as fragrances, food additives, and precursors for polymers and pharmaceuticals. The disclosed process further provides for purification of the phenyl alcohols using a technique less complicated than a simulated moving bed alternative. The disclosed process also addresses a wide variety of carbon sources suitable for microbial gas fermentation that converts carbon sources into valuable products such as phenyl alcohols that would otherwise be discarded to one or more products.

It is to be understood that the terminology used herein is for the purpose of describing particular embodiments only and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “carbon capture” as used herein refers to the fixation and utilization of carbon including carbon from CO₂, CO, and/or CH₄ from a stream comprising CO₂, CO, and/or CH₄ and converting the CO₂, CO, and/or CH₄ into useful products.

The term “substrate comprising carbon monoxide” and like terms should be understood to include any substrate in which carbon monoxide is available to one or more strains of bacteria for growth and/or fermentation, for example.

The term “gaseous substrates comprising carbon monoxide” includes any gas which contains carbon monoxide. The gaseous substrate will typically contain a significant proportion of CO, preferably at least about 5 vol.-% to about 100 vol.-% CO.

The term “C1 carbon” and like terms should be understood to refer to carbon sources that are suitable for use by a C1 fixing microorganism, particularly those of the gas fermentation process disclosed herein. C1 carbon may include, but should not be limited to, carbon monoxide (CO), carbon dioxide (CO₂), and methane (CH₄), methanol (CH₃OH), and formate (HCOOH).

The term “substrate comprising carbon dioxide” and like terms should be understood to include any substrate in which carbon dioxide is available to one or more strains of bacteria for growth and/or fermentation, for example.

The term “gaseous substrates comprising carbon dioxide” includes any gas which contains carbon dioxide. Some gaseous substrates contain a significant proportion of CO₂, such as at least about 50 vol.-% to about 100 vol.-% CO₂, while other gaseous substrates that contain CO₂ are more dilute and contain from about 5 vol.-% to about 50 vol.-% Advantageously the present disclosure reduces energy requirement for gaseous substrates comprising from about 5 vol.-% to about 20 vol.-% carbon dioxide thereby allowing dilute CO₂ containing streams to be processed economically by gas fermentation.

The term “bioreactor” includes a fermentation device consisting of one or more vessels and/or towers or piping arrangements, which includes the continuous stirred tank reactor (CSTR), immobilized cell reactor (ICR), trickle bed reactor (TBR), bubble column, gas lift fermenter, loop reactors, membrane reactor such as hollow fiber membrane bioreactor (HFMBR), static mixer, high throughput, or other vessel or other device suitable for gas-liquid contact.

The term “co-substrate” refers to a substance that, while not necessarily being the primary energy and material source for product synthesis, can be utilized for product synthesis when added to another substrate, such as the primary substrate.

The term “directly”, as used in relation to the passing of industrial off or gases to a bioreactor, is used to mean that no or minimal processing or treatment steps, such as cooling and particulate removal are performed on the gases prior to them entering the bioreactor (note: an oxygen removal step may be required for anaerobic fermentation).

The terms “fermenting,” “fermentation process,” “fermentation reaction,” and like terms as used herein, are intended to encompass both the growth phase and product biosynthesis phase of the process. As is described further herein, in some embodiments the bioreactor may comprise a primary bioreactor and a secondary bioreactor.

The term “nutrient medium” as used herein should be understood as the solution added to the fermentation broth containing nutrients and other components appropriate for the growth of the microorganism culture.

The terms “primary bioreactor” or “first reactor” as used herein this term is intended to encompass one or more reactors that may be connected in series or parallel with a secondary bioreactor. The primary bioreactors generally use anaerobic or aerobic fermentation to produce a product (e.g., ethylene, ethanol, acetate, etc.) from a gaseous substrate.

The terms “secondary bioreactor” or “second reactor” as used herein are intended to encompass any number of further bioreactors that may be connected in series or in parallel with the primary bioreactors. Any one or more of these further bioreactors may also be connected to a further separator.

The term “stream” is used to refer to a flow of material into, through and away from one or more stages of a process, for example, the material that is fed to a bioreactor. The composition of the stream may vary as it passes through particular stages. For example, as a stream passes through the bioreactor.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The term “underutilized gas” or “underutilized gas stream” may be used to refer to any gas stream that may have greater value as a substrate to gas fermentation than a current use.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

For many industrial processes, emission of gases that contain carbon are commonplace. Industrial process operators may view flaring and venting carbon rich sources to the atmosphere or otherwise discarding them as traditional standard techniques. Many domestic and international governmental entities are placing tighter restrictions on the total amount of waste carbon that a particular site, complex, or entity is allowed to release into the atmosphere. Such restrictions are pushing industrial, commercial, and agricultural operators alike to pursue and implement expensive efficiency upgrades to perhaps already well-developed technologies within their respective fields. Processes and systems in accordance with the present disclosure can be used to transform carbon in the gaseous feedstock into valuable products such as phenyl alcohols.

Processes and systems in accordance with the present disclosure can further be used to transform carbon in solid and liquid feedstocks by microbial fermentation systems, such as gas fermentation systems, to generate valuable products and divert carbon compounds from being treated as waste and for example incinerated to create emissions into the atmosphere.

The primary product of gas fermentation may be provided to downstream operations to produce articles of manufacture. The product of gas fermentation may be provided for use as an intermediate, a reactant, a solvent, an ingredient, or other such uses in the downstream operation.

Gas fermentation processes that are capable of converting various carbon sources into other products are rapidly becoming a desirable alternative for producers with excess carbon. Such processes allow companies or organizations to convert standard techniques that emit carbon into the atmosphere into a separate revenue stream by converting the waste or underutilized carbon into a marketable product. Moreover, the carbon that is converted into other products lowers the operator's total carbon output, potentially serving as a way for operators to maintain current outputs without conflicting with ever-tightening government regulations. Furthermore, tail gas from gas fermentation may be another source of CO₂ and purified to form a concentrated CO₂ stream thereby further reducing cost as compared to more traditional carbon capture and sequestration processes. The widespread adoption of gas fermentation processes could be improved by reducing the cost barriers through the use of additional material from the integrated process being available to downstream operations.

In one embodiment the substrate and/or C1 carbon source provides both the energy and the carbon source for the metabolic process of the biocatalyst, while in another embodiment, such as when CO₂ is the carbon source, depending upon the biocatalyst, a source of energy for the metabolic process is also provided. Typically, the source of energy for the metabolic process may be hydrogen. The hydrogen may be mixed with the C1 carbon source prior to the bioreactor of the gas fermentation system or may be independently supplied to the bioreactor.

The substrate and or C1 carbon source may be in the form of a solid or liquid material which may be first processed in a preliminary step of the overall integrated gas fermentation process to generate synthesis gas known as syngas which in turn is provided to the bioreactor of the gas fermentation system. The preliminary step to generate syngas may involve pyrolysis, partial oxidation, plasma, torrefaction, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material, and gasification of tires including gasification of end of life tires. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture waste and forest waste.

The microorganism of the disclosure may be cultured with the gaseous substrate to produce one or more target products. Target product(s) may be selected from an alcohol, an acid, a diacid, an alkene, a terpene, an isoprene, and alkyne. For instance, the microorganism of the disclosure may produce or may be engineered to produce ethanol, acetate, 1-butanol, 2,3-butanediol, lactate, butene, butadiene, methyl ethyl ketone (2-butanone), ethylene, acetone, isopropanol, lipids, 3-hydroxypropionate (3-HP), terpenes, including isoprene, fatty acids, 2-butanol, 1,2-propanediol, 1 propanol, 1 hexanol, 1 octanol, chorismate-derived products, 3 hydroxybutyrate, 1,3 butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, and/or monoethylene glycol in addition to ethylene. In certain embodiments, microbial biomass itself may be considered a product. An embodiment discussed in this disclosure includes wherein the microorganism of the disclosure is cultured with the gaseous substrate to produce one or more phenyl alcohols.

Examples of phenyl alcohols include, phenylethyl alcohol (also known as 2-phenylethanol), phenyl acetate, Benzyl alcohol-(phenyl-³C₆), 2-bromobenzyl alcohol-(phenyl-¹³C₆, 1-Phenylethanol, α-methylbenzyl alcohol, 2-phenyl-2-propanol, 2-phenyl-d₅-ethanol, dextroamphetamine, (R)-(+)-1-phenylethanol, 1-phenyl-2-propanol, 2-phenyl-1-propanol, 3-phenyl-1-propanol, (±)-α-(trifluoromethyl)benzyl alcohol, β-ethylphenethyl alcohol, 1-phenyl-1-propanol, cinnamyl alcohol, (S)-(−)-1-phenyl-1-propanol, (S)-(−)-1-phenyl-1-butanol, α-isobutylphenethyl alcohol, D-(−)-salicin, 1-phenyl-d₅-ethanol, (R)-1-phenyl-2-propyn-1-ol, 1,1,1,3,3,3-hexafluoro-2-phenyl-2-propanol, 4-acetoxybenzyl alcohol, salicin, 1-phenyl-2-propyn-1-ol, (S)-(+)-α-(trifluoromethyl)benzyl alcohol, (1S,2R)-(+)-norephedrine, 2-methyl-1-phenyl-2-propanol, (1R,2S)-(−)-ephedrine, terfenadine alcohol, (1S,2R)-(+)-ephedrine, ephedrine, diclofenac Impurity C, {2-[(2,6-dichlorophenyl)amino]phenyl}methanol, (S)-(−)-3-chloro-1-phenyl-1-propanol, (2RS)-2-[4-(2-methylpropyl)phenyl]propan-1-ol, phenyl isothiocyanate, phenoxyethanol, 2-phenoxyethanol, benzhydryl, 5-phenyl-1-pentanol, cyclizine impurity B, diphenylmethanol, ethylene glycol monophenyl ether, phenol, 4-phenyl-1-butanol, benzyl alcohol, 1-phenyl-1,2-ethanediol, 2-amino-1-phenylethanol, cinnamyl acetate, benzyl cinnamate, 1-phenyl-2-(1-piperidinyl)ethanol, 1,1-diphenyl-2-propyn-1-ol, α,α-dimethylbenzenepropanol, 2-hydroxyacetophenone, 2-phenyl-3-butyn-2-ol, (±)-norephedrine-d₃, (1S,2S)-(+)-Pseudoephedrine-d₃, phenylpropanolamine, R-(−)-phenylephrine, (+)-norpseudoephedrine, (±)-norpseudoephedrine-D₃, (±)-phenylpropanolamine, (1S,2R)-(+)-ephedrine-d₃, DL-norephedrine hydrochloride, and 2-(N-ethylaniline)ethanol.

For ease of understanding the disclosure is discussed with reference to 2-PE as the target metabolite of interest.

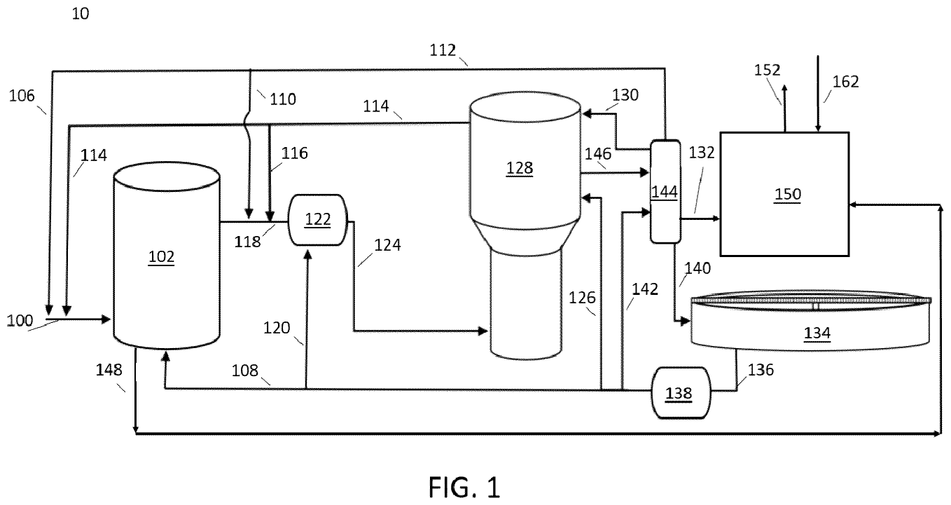

Referring now to FIG. 1, which illustrates an exemplary integrated gas fermentation (GF) system 10 having a feedstock preparation zone within the system 10. FIG. 1 shows an enlarged gas fermentation process including the feedstock preparation as gasification process 102, a gas fermentation zone 128, a product recovery zone 144, and an optional wastewater treatment zone 134, and a downstream operation to produce an article of manufacture 150. Feedstock preparation gasification process 102 receives feedstock 100, which may be any suitable material capable of being gasified to produce syngas stream 118. In various instances, feedstock 100 may be comprised at least partially of sorted and/or unsorted industrial or municipal solid waste including tires and end of life tires. In other instances, the feedstock 100 is comprised at least partially of forest and/or agricultural waste. In particular embodiments, feedstock 100 is comprised of any combination of two or more of the following: sorted municipal or industrial solid waste, unsorted municipal or industrial solid waste, tires including end of life tires, rubber containing material, forest waste, agricultural waste, or other solid or liquid waste from the refining or chemical process integrated with the enlarged gas fermentation process. In particular embodiments, a combination of two or more materials are processed together as one material alone may be difficult to process. Integration internal to the enlarged fermentation process may provide for at least one effluent from the gas fermentation zone 128, at least one effluent from the product recovery zone 144, and or at least one effluent from the wastewater treatment zone 134 being used as gasification feed.

Gasification zone 102 produces syngas as substrate for gas fermentation zone 128. In some embodiments, syngas 118 produced in the gasification zone 102 by the gasification process, or gas obtained from another source and combined with the syngas 118 contains one or more constituent that needs to be removed and/or converted. Typical constituents found in the syngas stream 118 that may need to be removed and/or converted include, but are not limited to, sulfur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars. These constituents may be removed by one or more removal zones 122 positioned between gasification zone 102a and gas fermentation zone 128. Removal zone 122 may comprise one or more of the following modules: hydrolysis module, acid gas removal module, deoxygenation module, catalytic hydrogenation module, particulate removal module, chloride removal module, tar removal module, and hydrogen cyanide polishing module. Two or more modules may be combined into a single module performing the same functions. The functions of all modules may be combined into a single unit with the selection of an appropriate catalyst, such as for example U.S. Pat. No. 11,441,116. When incorporating removal process 122, at least a portion of syngas 118 from gasification zone 102a is passed to removal process 122 to remove and/or convert at least a portion of at least one constituent found in syngas stream 118. Removal zone 122 may operate to bring the constituent(s) within allowable levels to produce a treated stream 124 suitable for fermentation by in gas fermentation zone 128.

Fermentation process 128 employs at least one C1-fixing microorganism in a liquid nutrient media to ferment a feedstock gas, or syngas stream 124 and produce one or more products. The C1-fixing microorganism in fermentation process 128 may be a carboxydotrophic bacterium, or an acetogenic carboxydotrophic bacterium. In particular embodiments, the C1-fixing microorganism may be an acetogenic carboxydotrophic bacterium. The C1-fixing microorganism may be selected from the group comprising Moorella, Clostridium, Ruminococcus, Acetobacterium, Eubacterium, Butyribacterium, Oxobacter, Methanosarcina, Methanosarcina, Cupriavidus and Desulfotomaculum. In various embodiments, the acetogenic carboxydotrophic bacterium is Clostridium autoethanogenum.

The one or more products produced in fermentation zone 128 are removed and/or separated from the fermentation broth in product recovery zone 144 (discussed in detail below). Product recovery zone 144 separates and removes one or more product(s) 132 and produces at least one effluent 142, 130, 112, which comprise reduced amounts of at least one product. Product depleted effluent may be sent via a conduit 142 to wastewater treatment zone 134 to produce at least one effluent 136, which may be recycled to the gasification process 102a and/or the fermentation process 128.

In at least one embodiment, an effluent from fermentation zone 128 is tail gas containing gas generated by the fermentation, inert gas, and or unmetabolized substrate. At least a portion of this tail gas may be passed via a conduit 114 to gasification zone 102 to be used as part of feedstock 100. At least a portion of the tail gas may be sent via conduit 114 and a conduit 116 to syngas 118, an effluent of gasification zone 102, to quench syngas stream 118. At least a portion of the tail gas may be passed outside of the enlarged gas fermentation process.

In at least one embodiment, the effluent from fermentation zone 128 is fermentation broth. At least a portion of the fermentation broth may be sent via conduit 146 to product recovery zone 144. In at least one embodiment, product recovery zone 144 separates at least a portion of the microbial biomass from the fermentation broth. In various instances, at least a portion of the microbial biomass that is separated from the fermentation broth is recycled to the fermentation zone 128 via a conduit 130. In various instances, at least a portion of the microbial biomass separated from the fermentation broth is passed via a conduit 106 to optional gasification zone 102 for use as part of feedstock 100. In certain instances, fermentation zone 128 produces fusel oil which may also be recovered in product recovery zone 144 through any suitable means such as within the rectification column of a distillation system. In at least one embodiment, at least a portion of the fusel oil from the product recovery zone 144 is used as a heating source for one or more zones or elsewhere external to the enlarged process.

In various instances, at least a portion of a wastewater stream, comprising fermentation broth, which may contain microbial biomass from fermentation zone 128 may be passed to optional gasification zone 102, without being passed to product recovery zone 144.

In instances where the fermentation broth is processed by the product recovery process 144, at least a portion of the microbial biomass depleted water, produced through the removal of microbial biomass from the fermentation broth, may be returned to fermentation zone 128 via a conduit 130 and/or sent via a conduit 112 to optional gasification zone 102a. At least a portion of the microbial biomass depleted water may be passed via conduit 106 to optional gasification zone 102a to be used as part of feedstock 100. At least a portion of the microbial biomass depleted water may be passed via conduit 110 to quench syngas stream 118. At least a portion of the effluent from product recovery zone 144 may be passed via conduit 140 to wastewater treatment zone 134. The effluent from product recovery zone 144 may comprise reduced amounts of product and/or microbial biomass.

Wastewater treatment zone 134 receives and treats effluent from one or more zones to produce clarified water. The clarified water may be passed or recycled via a conduit 136 to one or more zones. For example, at least a portion of the clarified water may be passed via conduit 126 to the fermentation zone, at least a portion of the clarified water may be passed to optional gasification zone 102 via conduit 108 to be used as part of feedstock 100 and or via conduit 120 to quench syngas stream 118 in quench zone 122. In certain instances, the wastewater treatment process 134 generates microbial biomass as part of the treatment process. At least a portion of this microbial biomass may be passed via conduit 108 to the gasification zone 102 for use as part of feedstock 100. Wastewater treatment zone 134, as a by-product of treating microbial biomass, produces biogas. At least a portion of the biogas may be passed via conduit 136 to gasification zone 102a to be used as part of feedstock 100 and or via a conduit 120 to quench syngas stream 118.

Optional wastewater treatment effluent removal unit 138 is positioned downstream of wastewater treatment zone 134. At least a portion of biogas from wastewater treatment zone 134 may be passed to removal unit 138 to remove and/or convert at least a portion of at least one constituent found in the biogas stream. Removal unit 138 operates to lower the concentration of constituents to within predetermined allowable levels and produce a treated stream 142, 126, 120, and/or 108 suitable to be used by the subsequent one or more zones 144, 128, 122, and/or 102a, respectively.

Gas fermentation product 132 is provided to downstream operation 150. Downstream operation 150 is an operation to produce an article of manufacture 152. Other feedstock(s) 162 may be provided to downstream operation 150. Gas fermentation product 132 may be a reactant, an intermediate, an ingredient, a production aide, or of other use in downstream operation 150. Feedstock preparation, shown as gasification 102, produces one or more byproducts 148. The one or more by products 148 are also provided for use in downstream operation 150.

Downstream operation 150 may involve one or more catalytic process steps in a catalytic process unit which can be a device consisting of one or more vessels and/or towers or piping arrangements, which includes the continuous stirred tank reactor (CSTR), immobilized cell reactor (ICR), trickle bed reactor (TBR), bubble column, gas lift fermenter, membrane reactor such as hollow fiber membrane bioreactor (HFMBR), static mixer, or other vessel or other device suitable for gas-liquid contact. Fixed bed, moving bed, simulated moving bed, fluidized bed, entrained bed, slurry reactor, packed bed, trickle bed, batch, semi batch, continuous, plug flow, flash, dense phase, fixed bed, downflow fixed bed, upflow expanded bed, and ebullating bed.

The types of catalysts used in the catalytic process unit can include, but are not limited to, natural clays, supported or unsupported metal or metal oxide containing catalysts, acid catalysts, zeolites, organometallic compounds. Examples include activated natural or synthetic material including activated, such as acid treated, natural clays such as bentonite type of synthesized silica-alumina or silica-magnesia, optionally with added oxides of zirconium, boron or thorium; mixed metal oxides supported on alumina or silica, such as tungsten-nickel sulfide or cobalt; metal and mixed metals containing catalysts such as platinum, palladium, rhenium, rhodium, copper, nickel, optionally supported on a silica or silica-alumina base; aluminum chloride, hydrogen chloride, sulfuric acid, hydrogen fluoride, phosphates, liquid phosphoric acid, phosphoric acid on kieselguhr, copper pyrophosphate pellets, phosphoric acid film on quarts, aluminosilicates, iron, vanadium, vanadium oxide on silica, nickel, silicone dioxide, carbonic anhydrase, iodine, zeolites, silver on alumina, Ziegler-Natta catalysts, organometallic compounds, iron oxide stabilized by chromium oxide, copper, copper-zinc-alumina, promoted iron where the promoters can be potassium oxide, aluminum oxide, and calcium oxide, and iron-chrome.

The products of gas fermentation can be catalytically converted, for example, by catalytic process unit. Additionally or alternatively, the products of gas fermentation can be catalytically converted, for example, by catalytically upgrading, into molecules, or one or more second products, wherein the one or more second products are integrated into existing or newly built infrastructure or feedstock and product transportation networks. Thus, in some embodiments, molecules produced via the catalysis of the products of gas fermentation processes may also be considered desirable products or further products of fermentation. For example, in a gas fermentation system that produces ethanol, that ethanol can reacted into a range of molecules, such as propane and benzene, toluene, ethylbenzene, xylene (BTEX), and these propane and BTEX molecules can be directly introduced into the feedstock or the existing product transportation networks/pipelines.

Phenyl alcohols have may uses such as food additives, pharmaceuticals, and monomers for polymers. Some phenyl alcohols have a pleasant scent and are used as additives in consumer products where fragrance is desired. Examples include perfume, household cleaners, cosmetics, lotions, oils, personal care items, car care, pet care, and the like. The fermentation produced phenol alcohols may be reacted to generate another compound that is used in articles of manufacture. Examples in the food additive area include converting a fermentation product of benzyl alcohol into benzaldehyde for use as almond flavoring and scents, converting fermentation product 2-PE into phenylacetaldehyde which may then be used as a fragrance, and converting fermentation produced 1-phenylethanol (1-PE) into acetophenone which may then be used as a fragrance. In the pharmaceuticals area fermentation product 1-phenyl-2-propanol may be converted into phenylacetone for use as amphetamine, methamphetamine; fermentation product 3-phenylpropanol may be converted into antibiotic potentiator and other organic compounds; and fermentation product 2-PE may be converted to phenylacetaldehyde and used as an antibiotic. In the polymer area, fermentation product 2-PE and or 1-PE may be converted into styrene and the styrene may be converted into acetonitrile butadiene styrene (ABS), styrene-butadiene rubber (SBR), styrene-isoprene-styrene (SIS), styrene acrylonitrile resin (SAN) and or styrene-divinylbenzene (S-DVB); fermentation product 2-PE may be converted into phenylacetaldehyde which in turn may be converted into polyesters. 1-PE fermentation product may be converted to acetophenone which is used in co-polymer resins for coatings and inks. ABS may be converted into plastics which is then used in a variety of articles. SBR may be used in the manufacture of tires. Note that tires, including end of life tires, may be a feedstock to the fermentation process via gasification with tires being an ultimate article made from the gas fermentation product. Similarly, polyesters are used in fabrics and textiles which may be used as a feedstock to the fermentation process via gasification with the ultimate article of manufacture from the fermentation product being new fabrics and textiles. These examples demonstrate the circularity of carbon in tires, fabrics, and textiles.

Turning to product recovery zone 144, the disclosure provides for recovery, purification, and concentration of 2-PE by taking a side draw from a standard distillation column. A nonideal interaction between 2-PE, ethanol and water, surprisingly lead to a significant concentration of 2-PE on certain distillation trays. With the 2-PE concentrating in the column at a specific location, removal of 2-PE using a side draw located at the section of the column, such as the center one-third of the column, with the concentration of 2-PE allowed for recovery of a high concentration product stream. Then, in the high concentration product stream, the 2-PE phase separates from the aqueous phase into another phase, termed the organic phase, having concentrated 2-PE. Examples below show 2-PE concentration at a location within the distillation column both by modeling and actual experiments. Examples below also show the phase separation of concentrated 2-PE in an organic phase from a water phase water by modeling and actual experiments.

The operating pressure the distillation column plays a role in the concentration of the 2-PE within the distillation column. Suitable operating pressures for the distillation column include from about atmospheric pressure to about 50 bar, from about atmospheric pressure to about 15 bar, from about atmospheric pressure to about 3 bar. When low pressure steam is available to heat the reboiler of the distillation column, a desired temperature in the distillation column is more economically achievable. The temperature of the distillation column would be in accordance with the heat produced by low pressure steam, and the pressure of the column would be adjusted in accordance with the temperature of the column.

Removing 2-PE from a traditional distillation column in recovery zone 144 has a number of advantages. First, the 2-PE is concentrated in an amount of more than about 10-fold as compared to the initial fermentation effluent. For example, 2-PE in the fermentation effluent after removal of the biocatalyst may be about 0.25 wt. % which may be concentrated to approximately 3 wt. % to about 5 wt. % on a distillation tray or cluster of distillation trays. Second, any biocatalyst present and or other gas fermentation products in the gas fermentation effluent exit the distillation column in the bottoms stream or another stream, resulting in the concentrated 2-PE side draw stream to be substantially free of particulate matter without the need for additional ultra-filtration. As the majority of water is removed from the distillation column via the distillation bottoms stream, the volume of the side draw is significantly reduced as compared to the distillation bottoms stream which reduces the scale of downstream secondary purification units or other units. The need for complex and costly techniques such as SMB for 2-PE recovery and purification is eliminated.

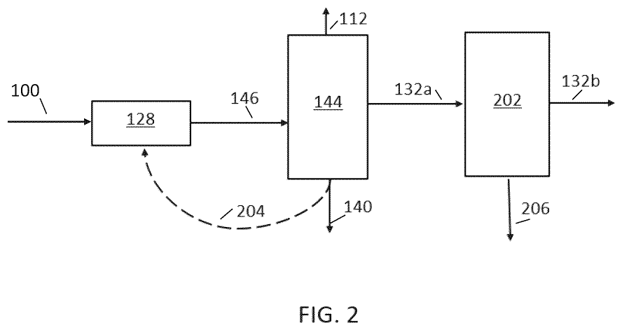

FIG. 2 illustrates a distillation column with a side draw of concentrated 2-PE. The location of the side draw is at a height of the column that is near to the inlet to the distillation column. The location of the side draw is in the center one-third of the column. FIG. 2 also shows separator unit 202 which allows for the phase separation of the aqueous phase and the organic phase to occur. The organic phase stream comprising the majority of the 2-PE of stream 132a is removed in stream 132b. An aqueous phase stream 206 is removed from unit 202. FIG. 2 also depicts an optional recycling of at least a portion of the distillation bottoms stream to the gas fermentation unit 128.

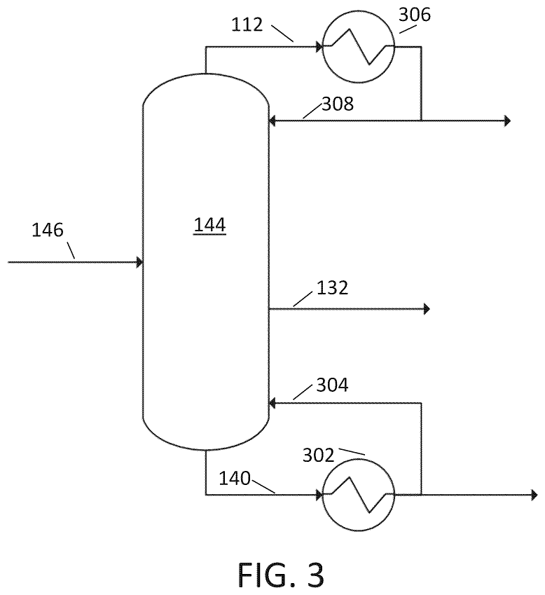

FIG. 3 illustrates more detail involving the distillation column 144. Fermentation broth in 146, either with biocatalyst or after biocatalyst has been removed, is passed to distillation column 144. 2-PE concentrates at a location within distillation column 144, and side draw stream 132 is located at a height of distillation column 144 in alignment with the concentration of 2-PE within the column to withdraw the concentrated 2-PE from the distillation column. FIG. 3 also show heat exchangers 302 and 306, and reboiler stream 304 and condensed stream 308.

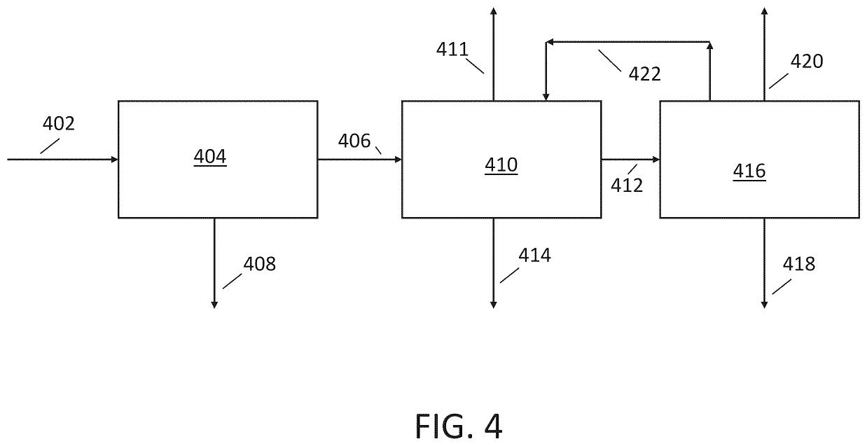

FIG. 4 illustrates one embodiment of the disclosure and a particular order of process units. In this embodiment, broth effluent 402 from the gas fermentation reactor system is passed to biocatalyst separation system 404. An exemplary system may be a filtration system or centrifuge system. Broth containing the biocatalyst is removed in broth recycle stream 408 and passed to the bioreactor system (not shown). Permeate 406 is removed from biocatalyst separation system 404 and passed to distillation column 410. Aqueous distillation bottoms 414 are removed from distillation column 410. Light byproducts such as perhaps ethanol are removed in overhead stream 411. Concentrated 2PE stream 412 is removed as a side draw from distillation column 410 and passed to phase separation unit 416. In phase separation unit 416, an aqueous phase separates from an organic phase wherein the 2-PE separates into the organic phase. Aqueous phase stream 420 is removed from phase separation unit 416. A portion of the aqueous phase may be recycled to distillation column 410 as recycle stream 422. Organic phase stream 418 comprising 2-PE is removed from phase separation unit 416.

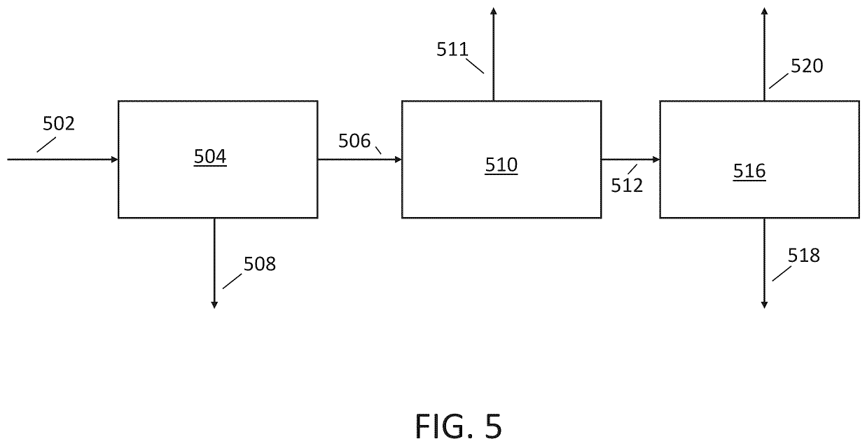

FIG. 5 illustrates one embodiment of the disclosure and a particular order of process units. In this embodiment, broth effluent 502 from the gas fermentation reactor system is passed to vacuum distillation biocatalyst separation system 504. Vacuum distillation bottoms 508 having aqueous broth depleted in fermentation product(s) from vacuum distillation unit 504 is recycled to bioreactor system (not shown). Distillate 506 containing fermentation product(s) is removed from vacuum distillation biocatalyst separation system 504 and passed to phase separation unit 510. In phase separation unit 510, an aqueous phase separates from an organic phase wherein the 2-PE separates into the organic phase. Aqueous phase stream 511 is removed from phase separation unit 510. Organic phase stream 512 comprising 2-PE is removed from phase separation unit 510 and passed to distillation column 516. As the bulk of water has been removed in the phase separation stage, the volume of water passed to distillation column 516 is significantly reduced. In distillation column 516, lighter boiling point components, as compared to 2-PE, such as water and other fermentation products such as ethanol are distilled to overhead 520 and removed. Distillation bottoms stream 518 comprising 2-PE is removed from distillation column 516.

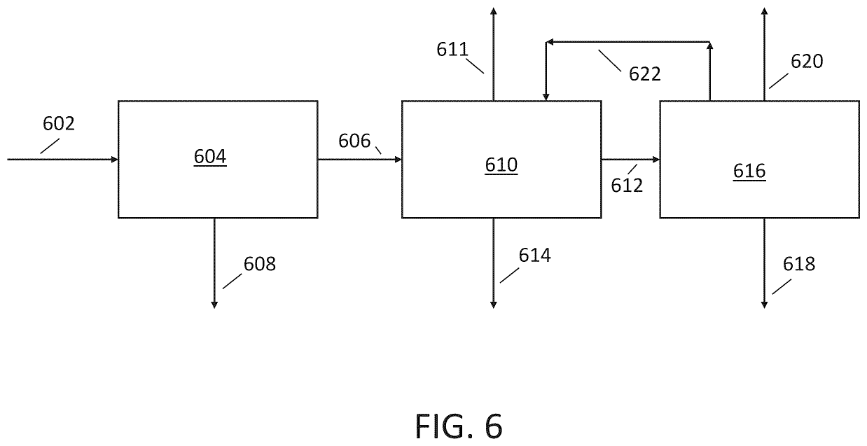

FIG. 6 illustrates one embodiment of the disclosure and a particular order of process units. illustrates one embodiment of the disclosure and a particular order of process units. In this embodiment, broth effluent 602 from the gas fermentation reactor system is passed to vacuum distillation biocatalyst separation system 604. Vacuum distillation bottoms 608 having aqueous broth depleted in fermentation product(s) from vacuum distillation unit 604 is recycled to bioreactor system (not shown). Distillate 606 containing fermentation product(s) is removed from vacuum distillation biocatalyst separation system 604 and passed to passed to distillation column 610. Aqueous distillation bottoms 614 are removed from distillation column 610. Light boiling byproducts, light as compared to 2-PE, such as perhaps ethanol are removed in distillation column overhead stream 611. Concentrated 2-PE stream 612 is removed as a side draw from distillation column 610 and passed to phase separation unit 616. In phase separation unit 616, an aqueous phase separates from an organic phase wherein the 2-PE separates into the organic phase. Aqueous phase stream 620 is removed from phase separation unit 616. Organic phase stream 618 comprising 2-PE is removed from phase separation unit 616. A portion of the aqueous phase may be optionally recycled to distillation column 610.

### Feedstock Preparation Byproducts

The disclosed systems and methods are also suitable for providing one or more feedstock preparation byproducts, referred to as secondary products, that are independent of the gas fermentation product (e.g., ethylene, ethanol, acetate, etc.). For example, in certain embodiments, microbial biomass itself may be considered a secondary product. In such embodiments, biomass from a bioreactor, such as dead microorganisms, may be used as a carbon source for further fermentation by gasifying the biomass. Additionally or alternatively, microbial proteins or other biomass may be recovered from a bioreactor and sold/used separately from the primary product (e.g., ethylene, ethanol, acetate, 1-butanol, etc.) as a supplement, such as a nutritional supplement and/or an animal feed. Known methods for using such biomass as a nutritional supplement or animal feed are disclosed in U.S. Pat. No. 10,856,560, which is herein incorporated by reference.

One or more byproducts of the feedstock preparation may be produced and recovered. One or more of the byproducts produced and recovered may be provided to the downstream operation. Two or more byproducts of the feedstock preparation may be produced and recovered and provided to the same downstream operation. The two or more byproducts may be provided to the same or different steps of the downstream operation. Three or more byproducts of the feedstock preparation may be produced and recovered and provided to the same downstream operation. The three or more byproducts may be provided to the same or different steps of the downstream operation. Four or more byproducts of the feedstock preparation may be produced and recovered and provided to the same downstream operation. The four or more byproducts may be provided to the same or different steps of the downstream operation. The byproducts of the feedstock preparation may be produced and recovered and provided to the same downstream operation to which the gas fermentation product is provided.

Additionally or alternatively, unutilized carbon dioxide, which may be in the form of an off-gas from the gas fermentation, may be a secondary product used within the gas fermentation process. Such unutilized carbon dioxide will be in a stoichiometrically higher proportion in the off-gas compared to the feedstock, and this relative purity can make the carbon dioxide useful. For example, the unutilized carbon can be sequestered by an operator for the purposes of obtaining carbon credits, or it may be combined with hydrogen gas (H₂), such as “green hydrogen” resulting from electrolysis, and recycled back into the gas fermenter or bioreactor as feedstock.

### Microorganisms and Fermentation

The disclosed systems and methods integrate microbial fermentation into existing or newly built infrastructure of, for example, a gas (e.g., natural gas) transportation pipeline, oil well, or the like to convert various feedstocks, gas, or other by-products into useful products such as ethylene. As disclosed herein, the systems allow for feedstocks, gas, or other by-products to be directly provided to a bioreactor, and the bioreactor is directly connected to a system for facilitating transport of a desirable product of fermentation to an end point (e.g., a chemical plant or refinery). In particular, the disclosed systems and methods are applicable for producing useful products (e.g., ethylene, ethanol, acetate, etc.) from gaseous substrates, such as gases that may optionally contain H₂, that are utilized as a carbon source by microbial cultures. Such microorganisms may include bacteria, archaea, algae, or fungi (e.g., yeast), and these classes of microorganism may be suitable for the disclosed systems and methods. In general, the selection of the microorganism(s) is not particularly limited so long as the microorganism is C1-fixing, carboxydotrophic, acetogenic, methanogenic, capable of Wood-Ljungdahl synthesis, a hydrogen oxidizer, autotrophic, chemolithoautotrophic, or any combination thereof. Among the various suitable classes of microorganisms, bacteria are particularly well suited for integration in the disclosed systems and methods.

When bacteria are utilized in the disclosed systems and methods, the bacteria may be aerobic or anaerobic, depending on the nature of the carbon source and other inputs being fed into the bioreactor or fermentation unit. Further, the bacteria utilized in the disclosed systems and methods can include one of more strains of carboxydotrophic bacteria. In particular embodiments, the carboxydotrophic bacterium can be selected from a genus including, but not limited to, Cupriavidus, Clostridium, Moorella, Carboxydothermus, Ruminococcus, Acetobacterium, Eubacterium, Butyribacterium, Oxobacter, Methanosarcina, Methanosarcina, and Desulfotomaculum. In particular embodiments, the carboxydotrophic bacterium is Clostridium autoethanogenum. In other particular embodiments, the carboxydotrophic bacterium is Cupriavidus necator.

A number of anaerobic bacteria are known to be capable of carrying out fermentation for the disclosed methods and system. Examples of such bacteria that are suitable for use in the disclosure include bacteria of the genus Clostridium, such as strains of Clostridium ljungdahlii (including those described in WO 00/68407, EP 117309, U.S. Pat. Nos. 5,173,429, 5,593,886, and 6,368,819, WO 98/00558 and WO 02/08438), Clostridium carboxydivorans (Liou et al., International Journal of Systematic and Evolutionary Microbiology 33: pp 2085-2091) and Clostridium autoethanogenum (Abrini et al., Archives of Microbiology 161: pp 345-351). Other suitable bacteria include those of the genus Moorella, including Moorella sp HUC22-1 (Sakai et al., Biotechnology Letters 29: pp 1607-1612), and those of the genus Carboxydothermus (Svetlichny, V. A., et al. (1991), Systematic and Applied Microbiology 14: 254-260). The disclosures of each of these publications are incorporated herein by reference. In addition, other carboxydotrophic anaerobic bacteria can be used in the disclosed systems and methods by a person of skill in the art. It will also be appreciated upon consideration of the instant disclosure that a mixed culture of two or more bacteria may be used in the disclosed systems and methods. All of the foregoing patents, patent applications, and non-patent literature are incorporated herein by reference in their entirety.

One exemplary anaerobic bacteria that is suitable for use in the disclosed systems and methods is Clostridium autoethanogenum. In some embodiments, the Clostridium autoethanogenum is a Clostridium autoethanogenum having the identifying characteristics of the strain deposited at the German Resource Centre for Biological Material (DSMZ) under the identifying deposit number 19630. In some embodiments, the Clostridium autoethanogenum is a Clostridium autoethanogenum having the identifying characteristics of DSMZ deposit number DSMZ 10061. In some embodiments, the Clostridium autoethanogenum is a Clostridium autoethanogenum having the identifying characteristics of DSMZ deposit number DSMZ 23693.

In some embodiments, the anaerobic bacteria is Clostridium carboxidivorans having the identifying characteristics of deposit number DSM15243. In some embodiments, the anaerobic bacteria is Clostridium drakei having the identifying characteristics of deposit number DSM12750. In some embodiments, the anaerobic bacteria is Clostridium ljungdahlii having the identifying characteristics of deposit number DSM13528. Other suitable Clostridium ljungdahlii strains may include those described in WO 00/68407, EP 117309, U.S. Pat. Nos. 5,173,429, 5,593,886, and 6,368,819, WO 98/00558 and WO 02/08438, all of which are incorporated herein by reference. In some embodiments, the anaerobic bacteria is Clostridium scatologenes having the identifying characteristics of deposit number DSM757. In some embodiments, the anaerobic bacteria is Clostridium ragsdalei having the identifying characteristics of deposit number ATCC BAA-622.

In some embodiments, the anaerobic bacteria is Acetobacterium woodii. In some embodiments, the anaerobic bacteria is from the genus Moorella, such as Moorella sp HUC22-1, (Sakai et al, Biotechnology Letters, 29: pp 1607-1612). Further examples of suitable anaerobic bacteria include, but are not limited to, Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp41-65). In addition, it should be understood that other C1-fixing, carboxydotrophic anaerobes may be suitable for the disclosed systems and methods. It will also be appreciated that a mixed culture of two or more bacteria may be utilized as well.

A number of aerobic bacteria are known to be capable of carrying out fermentation for the disclosed methods and system. Examples of such bacteria that are suitable for use in the disclosure include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

The fermentation may be carried out in any suitable bioreactor. In some embodiments, the bioreactor may comprise a first, growth reactor in which the microorganisms (e.g., bacteria) are cultured, and a second, fermentation reactor, to which fermentation broth from the growth reactor is fed and in which most of the fermentation product (e.g. ethylene, ethanol, acetate, etc.) is produced.

It will be appreciated that for growth of the bacteria and fermentation to occur, in addition to a carbon-containing substrate gas, a suitable liquid nutrient medium will need to be fed to the bioreactor. A nutrient medium will contain vitamins and minerals sufficient to permit growth of the micro-organism used. Aerobic and anaerobic media suitable for the fermentation using carbon-containing substrate gases as the sole carbon source are known in the art. For example, suitable media are described in U.S. Pat. Nos. 5,173,429, 5,593,886, WO 02/08438, WO2007/115157, and WO2008/115080, referred to above and all of which are incorporated herein by reference. Further, the fermentation can be carried out under appropriate conditions for the desired fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations, and maximum product concentrations to avoid product inhibition.

The optimum reaction conditions will depend partly on the particular micro-organism used. However, in general, it may be preferable that the fermentation be performed at a pressure higher than ambient pressure. Operating at increased pressures may allow for, for example, a significant increase in the rate of CO transfer from the gas phase to the liquid phase where it can be taken up by the micro-organism as a carbon source. This, in turn, means that the retention time (defined as the liquid volume in the bioreactor divided by the input gas flow rate) can be reduced when bioreactors are maintained at elevated pressure rather than atmospheric pressure. Also, since a given CO, or CO₂ and H₂ conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment.

Similarly, temperature of the culture may vary as needed. For example, in some embodiments, the fermentation is carried out at a temperature of about 34° C. to about 37° C. In some embodiments, the fermentation is carried out at a temperature of about 34° C. This temperature range may assist in supporting or increasing the efficiency of fermentation including, for example, maintaining or increasing the growth rate of bacteria, extending the period of growth of bacteria, maintaining or increasing production of the desired product (e.g., ethylene, ethanol, acetate, etc.), or maintaining or increasing CO or CO₂ uptake or consumption.

Culturing of the bacteria used in the disclosed systems and methods may be conducted using any number of processes known in the art for culturing and fermenting substrates. In some embodiments a culture of a bacterium can be maintained in an aqueous culture medium. For example, the aqueous culture medium may be a minimal anaerobic microbial growth medium. Suitable media are known in the art and described for example in U.S. Pat. Nos. 5,173,429 and 5,593,886; WO 02/08438, and in Klasson et al (1992), Bioconversion of Synthesis Gas into Liquid or Gaseous Fuels, Enz. Microb. Technol. 14:602-608; Najafpour and Younesi (2006) Ethanol and acetate synthesis from waste gas using batch culture of Clostridium ljungdahlii. Enzyme and Microbial Technology, 38(1-2):223-228; and Lewis et al (2002), Making the connection-conversion of biomass-generated producer gas to ethanol, Abst. Bioenergy, p. 2091-2094.

Further general processes for using gaseous substrates for fermentation that may be utilized for the disclosed systems and methods are described in the following disclosures: WO98/00558, M. Demler and D. Weuster-Botz (2010), Reaction Engineering Analysis of Hydrogenotrophic Production of Acetic Acid by Acetobacterium woodii, Biotechnology and Bioengineering; D. R. Martin, A. Misra and H. L. Drake (1985), Dissimilation of Carbon Monoxide to Acetic Acid by Glucose-Limited Cultures of Clostridium thermoaceticum, Applied and Environmental Microbiology, 49(6):1412-1417. Further processes generally described in the following articles using gaseous substrates for fermentation may also be utilized: (i) K. T. Klasson, et al. (1991), Bioreactors for synthesis gas fermentations resources, Conservation and Recycling, 5:145-165; (ii) K. T. Klasson, et al. (1991), Bioreactor design for synthesis gas fermentations, Fuel, 70:605-614; (iii) K. T. Klasson, et al. (1992), Bioconversion of synthesis gas into liquid or gaseous fuels, Enzyme and Microbial Technology, 14:602-608; (iv) J. L. Vega, et al. (1989), Study of Gaseous Substrate Fermentation: Carbon Monoxide Conversion to Acetate. 2. Continuous Culture, Biotech. Bioeng., 34(6):785-793; (vi) J. L. Vega, et al. (1989), Study of gaseous substrate fermentations: Carbon monoxide conversion to acetate. 1. Batch culture, Biotech. Bioeng., 34(6):774-784; (vii) J. L. Vega, et al. (1990), Design of Bioreactors for Coal Synthesis Gas Fermentations, Resources, Conservation and Recycling, 3:149-160; all of which are incorporated herein by reference.

As noted above, while bacteria may be preferred microorganisms for the disclosed systems and methods, other microorganisms like yeast may also be suitable. For example, yeast that may be used in the disclosed systems and methods include genus Cryptococcus, such as strains of Cryptococcus curvatus (also known as Candida curvatus) (see Chi et al. (2011), Oleaginous yeast Cryptococcus curvatus culture with dark fermentation hydrogen production effluent as feedstock for microbial lipid production, International Journal of Hydrogen Energy, 36:9542-9550, which is incorporated herein by reference). Other suitable yeasts include those of the genera Candida, Lipomyces, Rhodosporidium, Rhodotorula, Saccharomyces, and Yarrowia. In addition, it should be understood that the disclosed systems and methods may utilize a mixed culture of two or more yeasts. Additional fungi that may be suitable for the disclosed systems and methods include, but are not limited to, fungi selected from Blakeslea, Cryptococcus, Cunninghamella, Mortierella, Mucor, Phycomyces, Pythium, Thraustochytrium and Trichosporon. Culturing of yeast or other fungi may be conducted using any number of processes known in the art for culturing and fermenting substrates using yeasts or fungi.

Typically, fermentation is carried out in any suitable bioreactor, such as a continuous stirred tank reactor (CTSR), a bubble column reactor (BCR) or a trickle bed reactor (TBR). Also, in some embodiments, the bioreactor may comprise a first, growth reactor in which the micro-organisms are cultured, and a second, fermentation reactor, to which fermentation broth from the growth reactor is fed and in which most of the fermentation product (e.g., ethylene, ethanol, acetate, etc.) is produced.

The disclosed systems and method may comprise a primary bioreactor and a secondary bioreactor. The efficiency of the fermentation processes may be further improved by a further process of recycling a stream exiting the secondary bioreactor to at least one primary reactor. The stream exiting the secondary bioreactor may contain unused substrates, salts, and other nutrient components. By recycling the exit stream to a primary reactor, the cost of providing a continuous nutrient media to the primary reactor can be reduced. This recycling step has the further benefit of potentially reducing the water requirements of the continuous fermentation process. The stream exiting the bioreactor can optionally be treated before being passed back to a primary reactor. For example, because yeasts generally require oxygen for growth, any media recycled from a secondary bioreactor to a primary bioreactor may need to have all oxygen substantially removed, as any oxygen present in the primary bioreactor will be harmful to an anaerobic culture in the primary bioreactor. Therefore, the broth stream exiting the secondary bioreactor may be passed through an oxygen scrubber to remove substantially all of the oxygen prior to being passed to the primary reactor. In some embodiments, biomass from a bioreactor (e.g., a primary bioreactor, secondary bioreactor, or any combination thereof) may be separated and processed to recover one or more products.

In some embodiments, both anaerobic and aerobic gases can be used to feed separate cultures (e.g., an anaerobic culture and an aerobic culture) in two or more different bioreactors that are both integrated into the same process stream.

As disclosed herein, the feedstock gas stream providing a carbon source for the disclosed cultures is not particularly limited, so long as it contains a carbon source. C1 feedstocks comprising methane, carbon monoxide, carbon dioxide, or any combination thereof may be preferred. Optionally, H₂ may also be present in the feedstock. In some embodiments, the feedstock may comprise a gaseous substrates comprising substrate comprising carbon monoxide. In some embodiments, the feedstock may comprise a gaseous substrates comprising substrate comprising carbon dioxide. In some embodiments, the feedstock may comprise a gaseous substrates comprising substrate comprising both carbon monoxide and carbon dioxide. In some embodiments, the feedstock may comprise a gaseous substrates comprising carbon monoxide. In some embodiments, the feedstock may comprise a gaseous substrates comprising carbon dioxide. In some embodiments, the feedstock may comprise a gaseous substrates comprising carbon monoxide, carbon dioxide, or any combination thereof.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

The microorganism contained in the bioreactor may be cultured with the feedstock and produce one or more gas fermentation products. For instance, the microorganism may produce or may be engineered to produce ethanol (U.S. Pat. No. 7,972,824), acetate (U.S. Pat. No. 7,972,824), 1-butanol (U.S. Pat. Nos. 8,293,509, 9,359,611 and 9,738,875), butyrate (U.S. Pat. No. 8,293,509), 2,3-butanediol (U.S. Pat. Nos. 8,658,408 and 10,590,406), lactate (U.S. Pat. No. 8,900,836), butene (US2012/045807), butadiene (US 2012/045807), methyl ethyl ketone (2-butanone) (US 2012/045807 and U.S. Pat. No. 9,890,384), ethanol which is then converted to ethylene (US 2013/157,322), acetone (US 9,410, 130), isopropanol (U.S. Pat. No. 9,410,130), lipids (U.S. Pat. No. 9,068,202), 3-hydroxypropionate (3-HP) (U.S. Pat. No. 9,994,878), terpenes, including isoprene (U.S. Pat. No. 10,913,958), fatty acids (U.S. Pat. No. 9,347,076), 2-butanol (U.S. Pat. No. 9,890,384), 1,2-propanediol (U.S. Pat. No. 9,284,564), 1-propanol (U.S. Pat. No. 9,284,564), 1 hexanol (U.S. Pat. No. 9,738,875), 1-octanol (U.S. Pat. No. 9,738,875), chorismate-derived products (U.S. Pat. No. 10,174,303), 3-hydroxybutyrate (U.S. Pat. No. 9,738,875), 1,3-butanediol (U.S. Pat. No. 9,738,875), 2-hydroxyisobutyrate or 2-hydroxyisobutyric acid (U.S. Pat. No. 9,738,875), isobutylene (U.S. Pat. No. 9,738,875), adipic acid (U.S. Pat. No. 9,738,875), 1,3-hexanediol (U.S. Pat. No. 9,738,875), 3-methyl-2-butanol (U.S. Pat. No. 9,738,875), 2-buten-1-ol (U.S. Pat. No. 9,738,875), isovalerate (U.S. Pat. No. 9,738,875), isoamyl alcohol (U.S. Pat. No. 9,738,875), monoethylene glycol (U.S. Pat. No. 11,555,209), 2-phenylethanol (US 2021/0292732), ethylene (US 2023/407,271), and proteins (US 2023/407,362 and US 2023/407,271), and any combination thereof.

In an embodiment, the metabolite is a phenyl alcohol. For ease of understanding the disclosure is discussed with reference to 2-PE as the target metabolite of interest.

### Substrates and C1-Carbon Sources

A portion of the substrate and/or C1-carbon source may be a gas obtained as a by-product of an industrial process or from another source, such as combustion engine exhaust fumes, biogas, landfill gas, direct air capture, flaring, or from electrolysis. However, a portion of the substrate and/or C1-carbon source is syngas generated by pyrolysis, torrefaction, or gasification. In other words, carbon in solid or liquid materials may be recycled by pyrolysis, torrefaction, or gasification to generate syngas which is used as the substrate and/or C1-carbon source in gas fermentation. A portion of the substrate and/or C1-carbon source may be natural gas, carbon dioxide from conventional and unconventional gas production, and or a gas comprising methane. Gas fermentation processes are flexible and any of these substrate and/or C1-carbon sources may be employed.

In certain embodiments, the industrial process source of an optional portion of the substrate and/or C1 carbon source is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as natural gas, oil, or coal fired boilers or heaters, and gas turbine exhaust. Another example is the flaring of compounds such as at oil and gas production sites. In these embodiments, the substrate and/or C1-carbon source may be captured from the industrial process before it is emitted into the atmosphere, using any known method.

The substrate and/or C1-carbon source is synthesis gas known as syngas, which may be obtained from reforming, partial oxidation, plasma, or gasification processes. Examples of gasification processes include gasification of coal, gasification of refinery residues, gasification of petroleum coke, gasification of biomass, gasification of lignocellulosic material, gasification of wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material, gasification of rubber containing material, including portions of tires and whole tires. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Tires including end of file tires may be a feedstock. Examples of biomass may include lignocellulosic material and microbial biomass. Lignocellulosic material may include agriculture by-products, forest by-products, and some industrial by-products. Whole tires may be processed by pyrolysis to form syngas.

Biomass may be created as by-products of “nature-based solutions” (NBS) and thus natured-based solutions may provide feedstock to the gas fermentation process. Nature-based solutions is articulated by the European Commission as solutions inspired and supported by nature, which are cost-effective, simultaneously provide environmental, social, and economic benefits and help build resilience. Such solutions bring more, and more diverse, nature and natural features and processes into cities, landscapes, and seascapes, through locally adapted, resource-efficient, and systemic interventions. Nature-based solutions must benefit biodiversity and support the delivery of a range of ecosystem services. Through the use of NBS healthy, resilient, and diverse ecosystems (whether natural, managed, or newly created) can provide solutions for the benefit of both societies and overall biodiversity. Examples of nature-based solutions include natural climate solutions (conservation, restoration and improved land management that increase carbon storage or avoid greenhouse gas emissions in landscapes and wetlands across the globe), halting biodiversity loss, socio-economic impact efforts, habitat restoration, and health and wellness efforts with respect to air and water. Biomass produced through nature-based solutions may be used as feedstock to gas fermentation processes.

A portion of the substrate and/or C1-carbon source may be a gas stream comprising methane. Such a methane containing gas may be obtained from: fossil methane emissions such as during fracking, wastewater treatment, livestock, agriculture, and municipal solid waste landfills. It is also envisioned that the methane may be burned to produce electricity or heat and the C1 by-products may be used as the substrate or carbon source. The substrate and/or C1-carbon source may be a gas stream comprising natural gas.

### EXAMPLES

### Example 1

A gas fermentation effluent broth, after removal of biocatalyst, was intentionally spiked with 2.5 g/L of 2-PE and distilled. The spiked broth was distilled in non-steady operation and the distillation column bottoms was analyzed. Table 1 shows the concentration of 2-PE in the distillation bottoms unexpectedly decreased to 900 mg/L.

TABLE 1

| | Analyte | | | | | | |
|---|---|---|---|---|---|---|---|
| | | | | 1,2- | 2,3- | | |
| | | | Acetic | Propane- | Butane- | Phosphoric | Lactic |
| | 2-PE | Ethanol | Acid | diol | diol | Acid | Acid |
| | mg/L | g/L | g/L | mg/L | mg/L | g/L | g/L |
| Broth spiked | 2421 | 5.17 | 8.89 | 1.5 | 10.6 | 0.48 | 0.02 |
| with 2-PE | | | | | | | |
| Distillation | 956 | 5.79 | 7.47 | 2.7 | 35.2 | 0.43 | 0.03 |
| bottoms | | | | | | | |

### Example 2

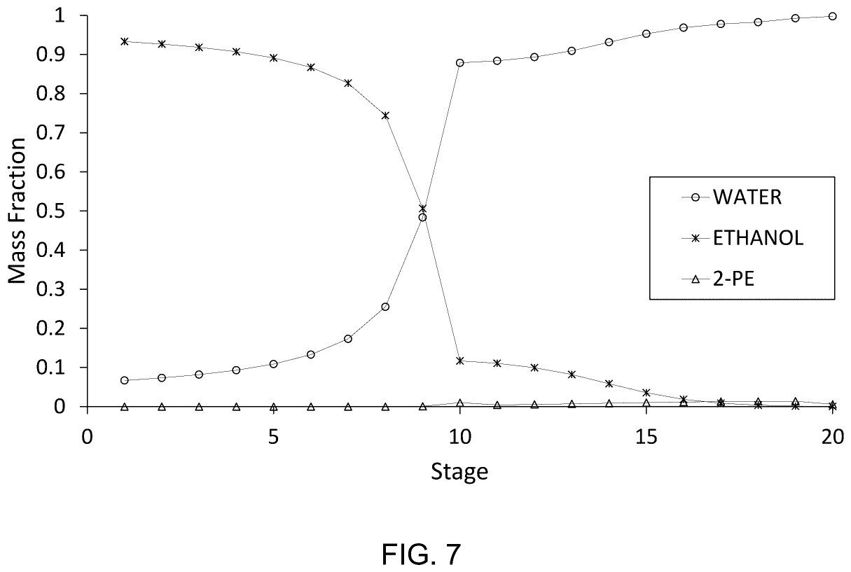

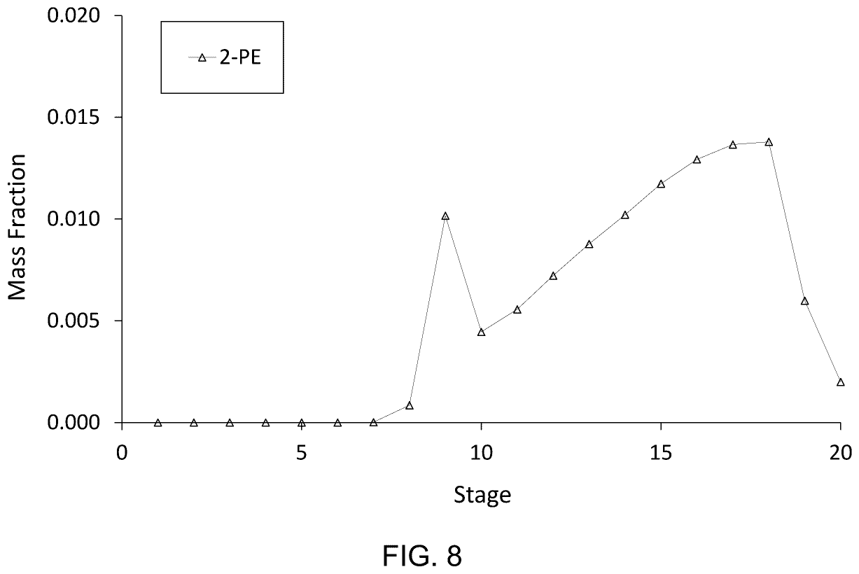

The distillation was modeled to investigate the concentration of 2-PE in the distillation bottoms being less than the amount of 2-PE intentionally added to the fermentation broth. The model was based on the concentration of 2-PE and ethanol as determined by analysis in Table 1 in a 20 stage distillation at a pressure of 1 bar. FIG. 7 show the results of the modeling and depict a column profile with the composition at each stage of the distillation column. The data show that the top one-half of the column comprised mainly ethanol while the bottom one-half of the column comprised water. However, the modeling showed that stage 9, which was the stage adjacent to and above the location of the distillation column feed input, and at stage 16, a concentration of 2-PE was indicated. Further modeling using Aspen software of a distillation column distilling a mixture of 2-PE, ethanol and water at the same conditions as above also shows stage 16 with greatly increased concentration of 2-PE and stage 9 with some increase in concentration of 2-PE. Results of the Aspen software modeling are shown in FIG. 8.

An azeotrope between 2-PE and water appeared to exist, and the presence of the binary azeotrope of 2-PE and water was confirmed by performing a batch distillation on a mixture of the two components. 21.3 g of 2-PE and 116.5 g of water were added to a distillation flask. The flask was connected to a spinning band distillation column, and the mixture was distilled at ambient pressure. Approximately the first 10 ml of distillate was collected and upon visual inspection a 2-phase mixture of 2-PE and water was observed. The presence of 2-PE in the distillate indicated a low boiling binary azeotrope of 2-PE and water. Without the presence of an azeotrope, the distillate would be expected to comprise only water with no 2-PE due to the large difference in boiling points, water having a boiling point of 100° C. and 2-PE having a boiling point of 220° C.

### Example 3

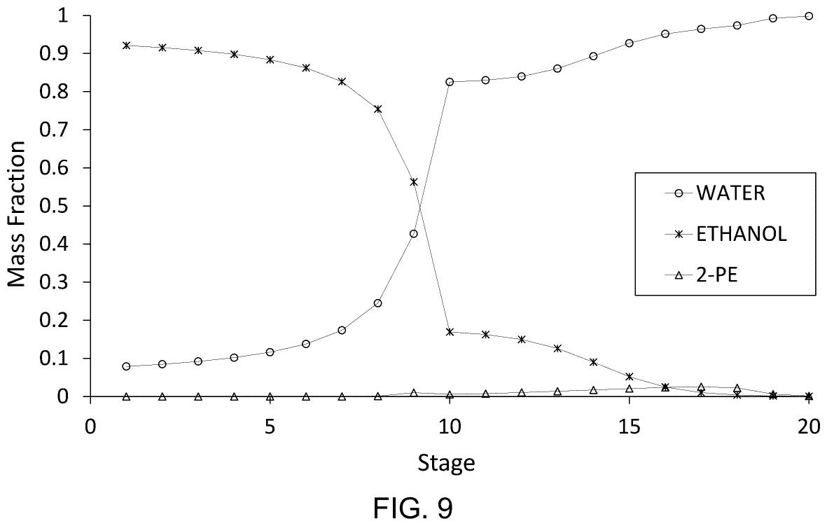

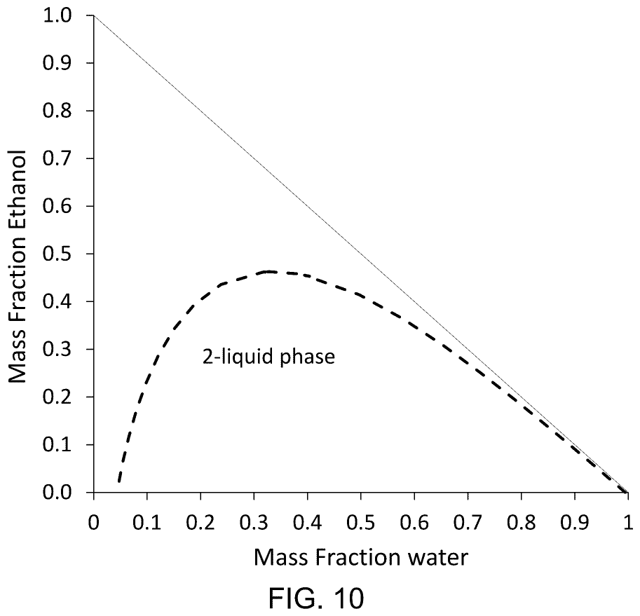

The same model as in Example 2 was repeated at a pressure of 3 bar. The resulting column profile for the separation of 2-PE and ethanol from water at 3 bar is shown in FIG. 9. At higher pressure than Example 2, the liquid composition on the middle trays were shown to be within the two-phase liquid-liquid boundary for the ternary mixture of ethanol, water, and 2-phenylethanol. The ternary diagram of 2-PE, ethanol, and water at 3 bar pressure was as shown in FIG. 10. A side draw removed at one or more of these tray locations provided a higher concentration 2-phenylethanol stream than the distillation bottoms produced at atmospheric pressure of Examples 1 and 2. Without being bound to theory, the higher pressure operation may affect the composition of the binary 2-PE and water azeotrope. Increasing the pressure may cause the 2-PE composition of the azeotrope to increase which results in an increased 2-PE composition on the trays around the side draw.

### Example 4

The same model as in Example 3 was repeated for a 20-tray distillation column at a pressure of 3 bar with the distillation column designed with a side draw as shown in FIG. 3. A total feed flowrate was selected to be 100 kg/hr for the simulation. The side draw was removed at distillation tray 16. The composition and total flow rates of the feed, bottoms, distillate overhead, and side draw are shown in Table 2. The results show that about 90 wt. % of the 2-PE in the feed was recovered in the side draw steam which in turn had a concentration of about 4.5 wt. % 2-PE.

TABLE 2

| | Units | Feed | Bottoms | Distillate | Side Draw |
|---|---|---|---|---|---|
| Mass Flows | | | | | |
| Water | kg/hr | 97.75 | 92.94 | 0.159 | 4.65 |
| Ethanol | kg/hr | 2.0 | 0.0352 | 1.84 | 0.124 |
| 2-PE | kg/hr | 0.25 | 0.0274 | 0.0 | 0.225 |
| Total | kg/hr | 100.0 | 93 | 2 | 5 |
| Mass Fraction | | | | | |
| Water | | 0.9775 | 0.999 | 0.0795 | 0.9307 |
| Ethanol | | 0.020 | 0.000378 | 0.921 | 0.0247 |
| 2-PE | | 0.0025 | 0.000295 | 0.0 | 0.0445 |

The simulated composition of the side draw stream lies within the liquid-liquid phase envelope and is therefore expected to separate into an organic layer with a 2-PE concentration close to 96 wt % and an aqueous layer with approximately 1 wt. % 2-PE.

### Example 5

Modeled phase separations above were confirmed by physical experiments using a mixture of about 2.2 wt. % ethanol, 5.5 wt. % 2-PE, and the balance being water. Upon initial mixing by centrifuge, one single phase was observed. The mixture was allowed to settle whereupon two phases were observed, the upper or lighter phase comprised water and the lower phase comprised 2-PE.

### Example 6

The simulation of Example 4 was repeated to compare the results when the solution modeled contains only 2-PE and water, as in Table 3, and when the solution modeled additionally contains other fermentation broth components, as in Table 4.

TABLE 3
Separation of 2-PE in water

| Species | Mass (g) | Mass Fraction |
|---|---|---|
| H₂O | 25.5 | 0.944 |
| 2-PE | 1.5 | 0.0556 |

TABLE 4
Separation of 2-PE and other Fermentation Products in water

| | Expected | Actual | Mass |
|---|---|---|---|
| Species | Mass (g) | Mass (g) | Fraction |
| H₂O | 25.5 | 25 | 0.909 |
| 2-PE | 1.5 | 1.5 | 0.0545 |
| Ethanol | 0.56 | 0.6 | 0.0218 |
| Acetic | 0.28 | 0.4 | 0.0145 |
| Acid | | | |

In the simulation, again a 20 try distillation column was modeled at 3 bar. 2-PE product stream was removed as a side draw at tray 16 and the side draw recovered greater than 90 wt. % of the 2-PE in the feed at 4.5 wt. % 2-PE. Expected phase separation of the side draw was confirmed by actual experiments using a centrifuge for mixing. A mixture of Table 4 was prepared and mixed by centrifuge. Upon settling, phase separation was observed. Surprisingly, after removal of an aqueous phase, two phases remained. All three phases were analyzed, and results are shown in Table 5.

TABLE 5

| | Acetic | | | |
|---|---|---|---|---|
| | Acid | Ethanol | 2-Phenylethanol | Dilution Factor |
| Analyte | g/L | g/L | g/L | RO Water |
| Phase 1 | 18.8 | 13.1 | 53.1 | 10x |
| Phase 2 | 18.62 | 12.8 | 29.5 | 10x |
| Phase 3 | Not | Not | Pure | 10x |
| | Detected | Detected | | |

Acetic acid is present in the feed at 1 wt % and is slightly concentrated in the side draw. The acetic acid can be removed from the side draw be treating with an appropriate resin prior to recovery of the 2-PE. Components 2,3-butanediol (BDO) and 1,2-propanediol (PDO) may be present in the feed in very low concentrations and may be present in the side draw at slightly lower concentrations as compared to the feed. Table 6 shows the results of the simulation of distillation as described above, 20 tray column at 3 bar, with side draw and the listed impurities.

TABLE 6

| | Units | Feed | Bottoms | Distillate | Side Draw |
|---|---|---|---|---|---|
| Mass Flows | | | | | |
| Water | kg/hr | 96.7 | 92.0 | 0.16 | 4.58 |
| Ethanol | kg/hr | 2.0 | 0.0254 | 1.84 | 0.132 |
| 2-PE | kg/hr | 0.25 | 0.0221 | 0.0 | 0.228 |
| Acetic Acid | kg/hr | 1.0 | 0.939 | 0.0 | 0.061 |
| 2,3-Butanediol | kg/hr | 0.004 | 0.004 | 0.0 | 0.0002 |
| 1,2- | kg/hr | 0.00003 | 0.00003 | 0.0 | 0.0 |
| Propanediol | | | | | |
| Total | kg/hr | 100.0 | 93 | 2 | 5 |
| Mass Fraction | | | | | |
| Water | | 0.967 | 0.989 | 0.0787 | 0.916 |
| Ethanol | | 0.02 | 0.0003 | 0.921 | 0.026 |
| 2-PE | | 0.0025 | 0.00024 | 0.0 | 0.0456 |
| Acetic Acid | | 0.01 | 0.01 | 0.0 | 0.012 |
| 2,3-Butanediol | | 4e−5 | 4e−5 | 0.0 | 3e−5 |
| 1,2- | | 3e−7 | 3e−7 | 0.0 | 2e−7 |
| Propanediol | | | | | |

Additionally or alternatively, a co-feed to the distillation column comprising an aqueous sodium hydroxide or potassium hydroxide solution can be used to remove acetic acid from the side draw. The co-feed would be introduced above the column feed inlet and would be relatively small compared to the flow rate of the primary column feed. The flow rate would be adjusted to introduce at least a stoichiometric ratio of the hydroxide salt to acetic acid, and could be increased to provide excess hydroxide salt to the acetic acid. The hydroxide salt would move down the column and react with acetic acid to form an acetate salt with no volatility. The salt would be removed from the column from the bottoms stream. In addition, the hydroxide salt will also remove any esters or thioesters from the permeate which may also help with downstream purification of the 2-PE.

### Example 7

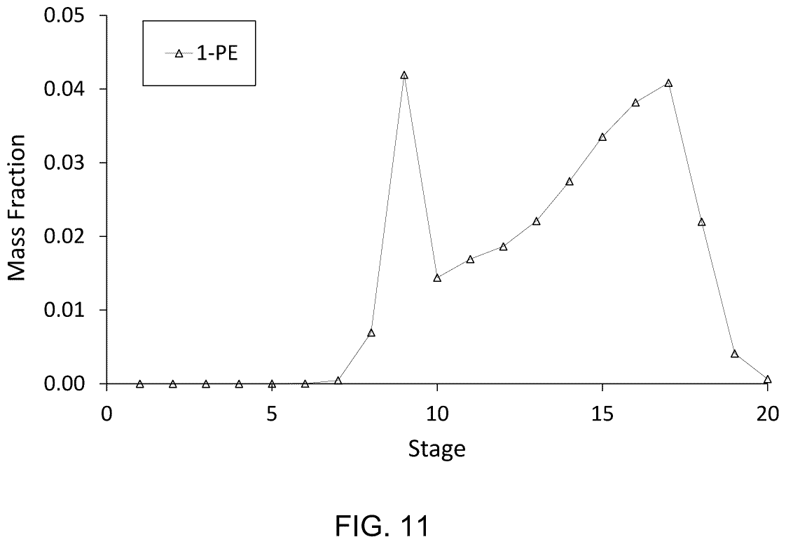

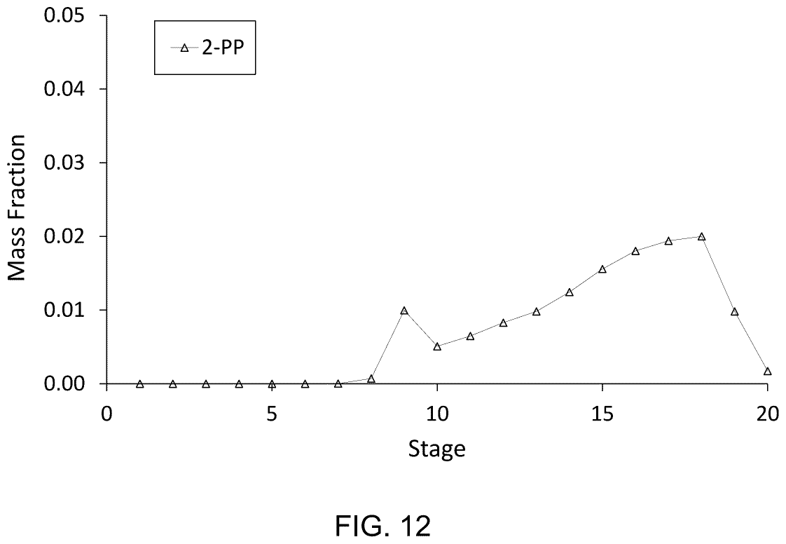

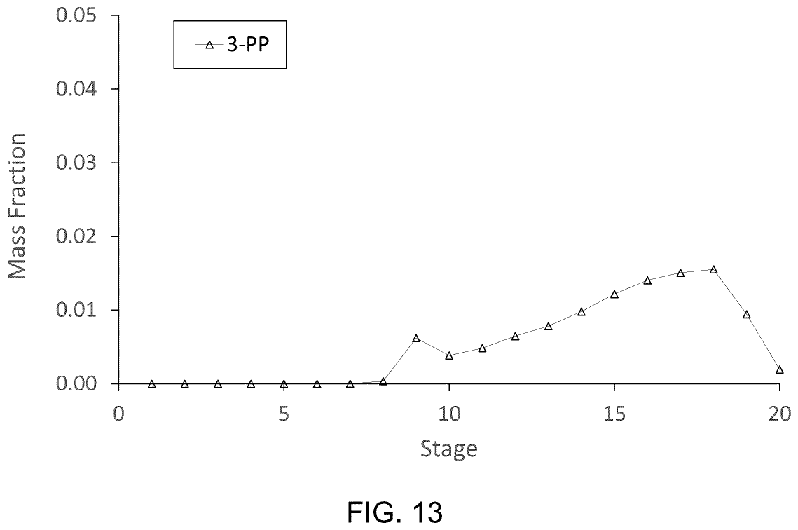

Simulated purification of other phenyl alcohols, 1-phenylethanol, 2-phenyl-1-propanol, 3-phenyl-1-propanol, in a rectification distillation column showed a similar concentration buildup of the phenyl alcohol within the distillation column. This concentration buildup is due to low boiling azeotropes formed between water and the individual phenyl alcohol. The column composition profile at 3 bar pressure for 1-phenylethanol (1-PE) is shown in FIG. 11, 2-phenyl-1-propanol (2-PP) is shown in FIGS. 12, and 3-phenyl-1-propanol (3-PP) is shown in FIG. 13.

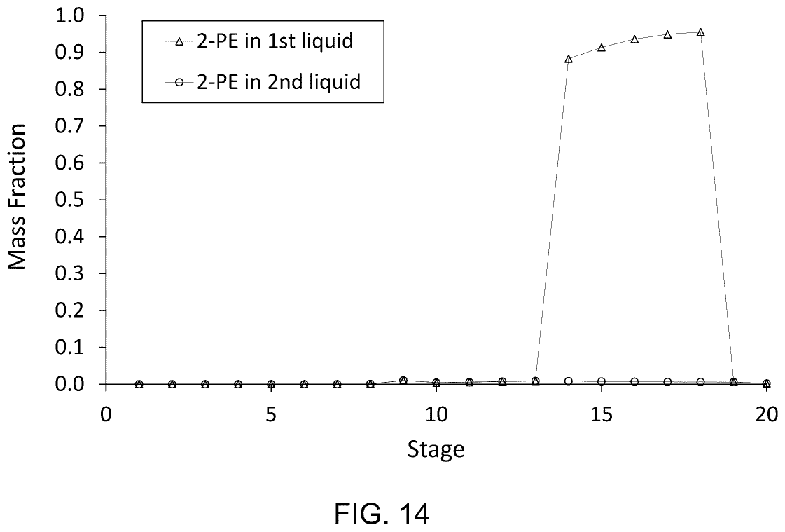

For each of 1-phenylethanol, 2-phenyl-1-propanol, 3-phenyl-1-propanol, and for 2-PE, a two phase liquid exists in the lower half of the column due to the low miscibility between water and the phenyl alcohols. A representative composition profile for the two liquid phases using 2-PE as an example is shown in FIG. 14. At stages 14-18, FIG. 14 shows a difference between the first liquid phase and the second liquid phase. In this case the first liquid phase is the organic phase that comprises substantially 2-PE. The second liquid phase is the aqueous phase that contains a low concentration of 2-PE. Stages where the first and second liquid phase are the same composition (1-13 and 19-20) and have only a single liquid phase present. Other phenyl alcohols have a similar 2-liquid phase region within the column although the position, number of stages, and phenyl alcohol concentration may change slightly with each phenyl alcohol.

The present disclosure is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology.

All patents, patent applications, and published references cited herein are hereby incorporated by reference in their entirety. The claims are intended to cover the components and steps in any sequence which is effective to meet the objectives there intended unless the context specifically indicates the contrary.

Unless the context requires otherwise, throughout this description and the claims, the words “comprise”, “comprising”, and the like, are to be construed in an inclusive sense as opposed to an exclusive or exhaustive sense, that is to say, in the sense of including, but not limited to.

The use of the terms “a” and “an” and “the” and similar terms are to be construed to cover both the singular and the plural, unless otherwise indicated herein or clearly contradicted by context. The terms “comprising,” “having,” “including,” and “containing” are to be construed as open-ended terms unless otherwise noted. The use of the alternative, such as the term “or”, should be understood to mean either one, both, or any combination thereof of the alternatives. Percentages are in wt. % unless otherwise stated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure, and does not pose a limitation on the scope of the disclosure unless otherwise claimed. No language in the specification should be construed as indicating any non-claimed element as essential to the practice of the disclosure.

Various embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### EMBODIMENTS OF THE DISCLOSURE

### Embodiment 1. A Process Comprising

a. generating a fermentation broth comprising microbial biomass, at least one phenyl alcohol fermentation product, at least one byproduct, and an aqueous liquid nutrient medium;
b. removing the microbial biomass from the fermentation broth to generate a microbial biomass-depleted stream;
c. distilling, in a distillation column, the microbial biomass-depleted stream and removing water in a distillation bottoms stream, the at least one byproduct in a distillation overhead stream, and the at least one phenyl alcohol in a distillation side draw stream;
d. settling the distillation side draw stream and forming at least two phases wherein a first phase comprises aqueous liquid nutrient medium and a second phase comprising phenyl alcohol; and
e. recovering the second phase comprising phenyl alcohol.

Embodiment 2. The process of embodiment 1 further comprising recycling at least a portion of the first phase to the distillation column.

Embodiment 3. The process of embodiment 1 or 2 wherein the microbial biomass is removed from the fermentation broth by filtration or vacuum distillation.

Embodiment 4. The process of any of embodiments 1 to 3 wherein the distillation bottoms stream is recycled to the generating a fermentation broth.

Embodiment 5. The process of any of embodiments 1 to 4 wherein the fermentation broth is generated by gas fermentation.

Embodiment 6. The process of any of embodiments 1 to 5 wherein the distillation is performed at a pressure above atmospheric pressure to 50, or from about atmospheric pressure to about 15 bar, or from about atmospheric pressure to about 3 bar.

Embodiment 7. The process of any of embodiments 1 to 6 wherein the side draw is located in the middle one third of the distillation column.

Embodiment 8. The process of any of embodiments 1 to 7 wherein the phenyl alcohol is 2-phenylethanol.

Embodiment 9. A process comprising:
a. generating a fermentation broth comprising microbial biomass, at least one phenyl alcohol fermentation product, at least one byproduct, and an aqueous liquid nutrient medium;
b. removing the microbial biomass from the fermentation broth by vacuum distillation to generate a microbial biomass-depleted distillate;
c. settling the microbial biomass-depleted distillate and forming at least two phases wherein a first phase comprises aqueous liquid nutrient medium and a second phase comprising phenyl alcohol; and
d. distilling, in a distillation column, the second phase comprising phenyl alcohol and removing water in a distillation bottoms stream, the at least one byproduct in a distillation overhead stream, and the at least one phenyl alcohol in a distillation bottoms stream.

Embodiment 10. The process of embodiment 9 wherein the microbial biomass is removed from the fermentation broth by filtration or vacuum distillation.

Embodiment 11. The process of embodiment 9 or 10 wherein the fermentation broth is generated by gas fermentation.

Embodiment 12. The process of any of embodiments 9 to 11 wherein the distillation is performed at a pressure above atmospheric pressure to about 50, or from about atmospheric pressure to about 15 bar, or from about atmospheric pressure to about 3 bar.

Embodiment 13. The process of any of embodiments 9 to 12 wherein the side draw is located in the middle one third of the distillation column.

Embodiment 14. The process of any of embodiments 9 to 13 wherein the phenyl alcohol is 2-phenylethanol.

1. A process comprising:
a) generating a fermentation broth comprising microbial biomass, at least one phenyl alcohol fermentation product, at least one byproduct, and an aqueous liquid nutrient medium;
b) removing the microbial biomass from the fermentation broth to generate a microbial biomass-depleted stream;
c) distilling, in a distillation column, the microbial biomass-depleted stream and removing water in a distillation bottoms stream, the at least one byproduct in a distillation overhead stream, and the at least one phenyl alcohol in a distillation side draw stream;
d) settling the distillation side draw stream and forming at least two phases wherein a first phase comprises aqueous liquid nutrient medium and a second phase comprising phenyl alcohol; and
e) recovering the second phase comprising phenyl alcohol.

2. The process of claim 1 further comprising recycling at least a portion of the first phase to the distillation column.

3. The process of claim 1 wherein the microbial biomass is removed from the fermentation broth by filtration or vacuum distillation.

4. The process of claim 1 wherein the distillation bottoms stream is recycled to the generating a fermentation broth.

5. The process of claim 1 wherein the fermentation broth is generated by gas fermentation.

6. The process of claim 1 wherein the distillation is performed at a pressure above atmospheric pressure to 50, or from about atmospheric pressure to about 15 bar, or from about atmospheric pressure to about 3 bar.

7. The process of claim 1 wherein the side draw is located in the middle one third of the distillation column.

8. The process of claim 1 wherein the phenyl alcohol is 2-phenylethanol.

9. A process comprising:
a) generating a fermentation broth comprising microbial biomass, at least one phenyl alcohol fermentation product, at least one byproduct, and an aqueous liquid nutrient medium;
b) removing the microbial biomass from the fermentation broth by filtration or vacuum distillation to generate a microbial biomass-depleted distillate;
c) settling the microbial biomass-depleted distillate and forming at least two phases wherein a first phase comprises aqueous liquid nutrient medium and a second phase comprising phenyl alcohol; and
d) distilling, in a distillation column, the second phase comprising phenyl alcohol and removing water in a distillation bottoms stream, the at least one byproduct in a distillation overhead stream, and the at least one phenyl alcohol in a distillation side draw stream.

10. (canceled)

11. The process of claim 9 wherein the fermentation broth is generated by gas fermentation.

12. The process of claim 9 wherein the distillation is performed at a pressure above atmospheric pressure to about 50, or from about atmospheric pressure to about 15 bar, or from about atmospheric pressure to about 3 bar.

13. The process of claim 9 wherein the side draw is located in the middle one third of the distillation column.

14. The process of claim 9 wherein the phenyl alcohol is 2-phenylethanol.

15. The process of claim 1 wherein the second phase comprises at least 90 wt.-% of the phenyl alcohol of the microbial biomass-depleted stream.

16. The process of claim 1 wherein the 2-phenylethanol of the phenyl alcohol of the microbial biomass-depleted stream is concentrated to from about 3 wt.-% to about 5 wt.-% on a distillation tray or cluster of distillation trays of the distillation column.

17. The process of claim 1 wherein the distillation side draw stream comprises an azeotrope between the 2-phenylethanol and water.
